(19) United States
(12) Patent Application Publication
(10) Pub. No.: US 2016/0023979 A1
(43) Pub. Date: Jan. 28, 2016

(54) PROCESS FOR THE ETHERIFICATION OF A POLYOL

(71) Applicant: SAUDI BASIC INDUSTRIES CORPORATION, Riyadh (SA)

(72) Inventor: Thomas Hubertus Maria HOUSMANS, Geleen (NL)

(21) Appl. No.: 14/772,478

(22) PCT Filed: Mar. 4, 2014
(86) PCT No.: PCT/IB2014/059432

§ 371 Date: Sep. 3, 2015

(30) Foreign Application Priority Data
Mar. 7, 2013 (EP) 13001152.1

(51) Int. Cl. C07C 41/06

(52) U.S. Cl. CPC C07C 41/06

(57) ABSTRACT

The present invention relates to a process for the production of polyol alkyl ethers by etherification of a diol or triol having 2-3 carbon atoms with a hydrocarbon having 2-10 carbon atoms selected from the group consisting of olefms, ketones and alcohols as alkylation agent in the presence of an etherification catalyst, wherein said process is performed in a tubular loop reactor comprising a polyol feed inlet, at least one alkylation agent feed inlet and a product outlet.

The present invention relates to an improved process for the production of polyol alkyl ethers by etherification of a diol or triol having 2-3 carbon atoms with a hydrocarbon having 2-10 carbon atoms selected from the group consisting of olefins, ketones and alcohols as alkylation agent in the presence of an etherification catalyst.

It has been previously described that polyol alkyl ethers can be produced from polyols. U.S. Pat. No. 5,731,476 discloses a process for the preparation of a polyether by reaction of a polyhydric compound having at least 3 hydrocyl groups per molecule with a C5-C10 tertiary olefin or a C4-C10 tertiary alkanol or ether or by reaction of isobutylene with a polyhydric compound having more than 3 hydroxyl groups per molecule in the presence of an acid catalyst. U.S. Pat. No. 5,476,971 discloses a process for the preparation of di-t-butyl glycerine by reaction of the hydrocarbon isobutylene with the polyhydric compound glycerine. The reactions of U.S. Pat. No. 5,731,476 and U.S. Pat. No. 5,476,971 are carried out in the liquid phase while maintaining separate phases comprised of a polar polyhydric compound phase and hydrocarbon phase.

DE 10 2009 055 928 A1 discloses a method for producing a higher glycerol tertiary butyl ether comprising mixture, comprising the steps of acid catalysed reaction of isobutene with glycerol to form a reaction mixture and the extraction of at least part of said reaction mixture using a solvent or solvent mixture having an E_{T}(30) polarity value of ≦35.0.

A major drawback of the processes of the prior art is that these have a relatively high selectivity for unwanted by-products such as mono- and/or tri-ethers and alkylation agent oligomerization products. Moreover, control over the product stream composition can only be achieved by a combination of the residence time in the reactor combined with separation and recycle of unreacted and intermediate products. Therefore relatively large recycle streams are required and the utility costs tend to be higher.

In a polyol etherification process, the hydroxyl groups of the polyol react with the alkylation agent to consecutively provide mono-, di- and poly-ethers. In order to improve the selectivity for di-ethers or poly-ethers, the concentration of the alkylation agent in the reaction mixture needs to be relatively high. However, such a relatively high concentration of the alkylation agent in the reaction mixture also leads to the formation of unwanted side products, such as oligomers or polymers of the alkylation agent. It is particularly difficult to obtain sufficient selectivity for di-ethers over the unwanted mono-ethers, poly-ethers and alkylation agent oligomerization products.

It was an object of the present invention to provide a process for the production of polyol alkyl di-ethers by etherification of a polyol which has an improved selectivity for di-ethers, without an increased selectivity for unwanted by-products such as alkylation agent oligomerization products.

The solution to the above problem is achieved by providing the embodiments as described herein below and as characterized in the claims. Accordingly, the present invention provides a process for the production of polyol alkyl ethers by etherification of a polyol in the presence of an etherification catalyst, wherein the process is performed in a tubular loop reactor (1) comprising a polyol feed inlet (2), an alkylation agent feed inlet (3) and a product outlet (4), wherein the polyol in the polyol feed is a diol or triol having 2-3 carbon atoms and the alkylation agent is a hydrocarbon having 2-10 carbon atoms selected from the group consisting of olefins, ketones and alcohols.

The polyol etherification processes according to the prior art are performed in continuous stirred-tank reactors (CSTR). By selecting the tubular loop reactor of the present invention, residence time and the molar ratio of the hydroxyl groups to alkylation agent in the reaction mixture can be instantly and precisely regulated. By injecting iso-butylene along the reactor and by adjusting the residence time, precise control over the concentration of the reactants can be achieved.

Since isobutene is only sparingly soluble in glycerol, the reactants are in separate liquid phases when the isobutene and glycerol are brought into contact. As the reaction proceeds the products accumulate in one or other of the phases according to their solubility. The mono-ethers accumulate in the polar glycerol phase and di-ethers, tri-ethers and C8-C16 hydrocarbons in the non-polar hydrocarbon phase. During the reaction the solubility changes: new product components are formed in the mixture and the concentrations of all the components changes. At some stage the two phases dissolve in each other and only one phase remains. This is believed to occur at a glycerol conversion of about 60-70%. By selecting a tubular reactor instead of a constant CSTR, the process can be operated in one or in a two phase regime. This means that the reactor effluent is either a single phase or a two phase which may be beneficial for easy separation of the different process products. Preferably, the process is operated under conditions that ensure maximum isobutene solubility.

Referring to the accompanying figures (referred to herein as Figure and FIG.), the present invention provides a process that is inter alia characterized in that a tubular loop reactor (1) is used. Tubular reactors are well known in the chemical industry and are generally described in Middleton, J. C. and Carpenter, K. J. (2010) Loop Reactors. Ullmann's Encyclopedia of Industrial Chemistry.

The tubular loop reactor used in the process of the present invention comprises at least one inlet (2) for feeding the polyol compound to the reactor.

Furthermore, the tubular loop reactor used in the present process comprises at least one inlet (3) for feeding the alkylating agent to the reactor. Preferably, the reactor comprises multiple alkylation agent feed inlets (3); see FIG. 2. By using multiple alkylation agent feed inlets, the concentration of alkylation agent in the reaction mixture can be controlled better, which allows tight control over the conversion and lowers the production of unwanted side-products. This embodiment of the present invention is particularly useful to keep the isobutylene:reactants molar ratio constant over the length of the reactor tube.

Preferably, the alkylation agent is added to the reaction mixture in the tubular loop reactor at a rate to provide a molar ratio of the hydroxyl groups to alkylation agent (molar ration of OH:alkylating agent) in the reaction mixture of 2:1 to 5:1. Maintaining the molar ratio of hydroxyl groups in the reaction mixture alkylating agent has the additional advantage of reduced a-specific reactivity of the alkylating agent, including, but not limited to, oligomerization of the alkylating agent.

The tubular loop reactor used in the process of the present invention comprises one product outlet (4) for removing the product from the reactor. The outlet may involve a solid-liquid-gas separator in case a heterogeneous catalyst is used.

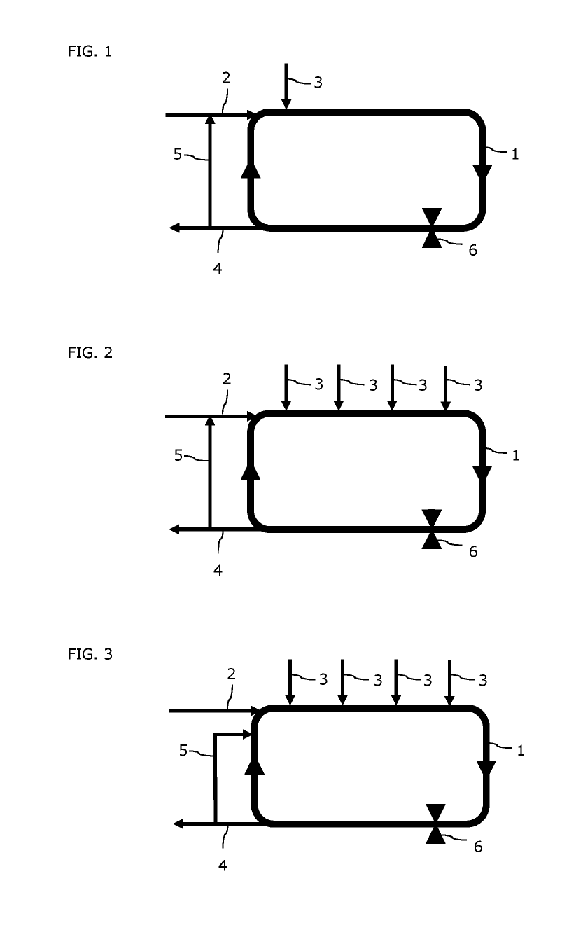

Optionally, the unreacted polyol compound or the unwanted mono-ethers comprised in the product stream (4) are separated from the desired polyol alkyl ethers and are recycled via recycle stream (5) to the inlet (2); see FIGS. 1 and 2, or directly to the reactor (1); see FIG. 3.

The reaction mixture is brought to recirculation inside the tubular loop reactor by an in-line pump (6) or other means.

Catalysts useful in the acid catalyzed condensation of alcohols are amply known in the art and include homogeneous acid catalyst and heterogeneous acid catalyst; see e.g. WO 2009/117044 A2 and Klepacova et al. (2007) Applied Catalysis A 328:1-13.

Preferably, the etherification catalyst is a homogeneous acid catalyst selected from the group consisting of sulfuric acid, sulfonic acid, and nitric acid. Preferably, the sulfonic acid is selected from the group consisting of trifluoromethanesulfonic acid, p-toluenesulfonic acid, methanesulfonic acid, ethanesulfonic acid, sulfosuccinic acid and sulfoacetic acid.

Preferably, the homogeneous acid catalyst residue comprised in the product outlet stream is neutralized and the resulting salt is separated from the product outlet stream.

Alternatively, the etherification catalyst is a porous heterogeneous acid catalyst selected from the group consisting of acidic large-pore zeolite, strong acid ion exchange resin and acid-functionalized mesostructured silicas. Such suitable acidic large-pore zeolites, strong acid ion exchange resins and acid-functionalized mesostructured silicas are well known in the art and are commercially available.

Preferably, the acidic large pore zeolite is selected from the group consisting of zeolite beta and zeolite Y. As used herein, the term “zeolite” or “aluminosilicate zeolite” relates to an aluminosilicate molecular sieve. An overview of their characteristics is for example provided by the chapter on Molecular Sieves in Kirk-Othmer Encyclopedia of Chemical Technology, Volume 16, p 811-853; in Atlas of Zeolite Framework Types, 5th edition, (Elsevier, 2001). The term “large pore sized zeolite” as used herein is very well-known in the art; see e.g. Holderich et al. (1988) Angew. Chem. Int. Ed. Engl. 27:226-246. Accordingly, a large pore size zeolite is a zeolite having a pore size of about 6-8 Å. Suitable medium pore size zeolites are 12-ring zeolites, i.e. the pore is formed by a ring consisting of 12 SiO4 tetrahedra. In the above cited Altlas of Zeolite Framework Types various zeolites are listed based on ring structure. Preferably, the silica (SiO₂) to alumina (Al₂O₃) molar ratio of the zeolite is in the range of about 300-1000. Zeolites having a silica to alumina molar ratio of 300-1000 are well known in the art and also are commercially available. Means and methods for quantifying the silica to alumina molar ratio of a zeolite are well known in the art and include, but are not limited to AAS (Atomic Absorption Spectrometer) or ICP (Inductively Coupled Plasma Spectrometry) analysis.

Preferably, the strong acidic ion exchange resin is selected from the group consisting of Amberlyst 15, Amberlyst 35 and Amberlyst XN1010.

The heterogeneous catalyst may be packed in a fixed bed inside the tubular loop reactor (1). Alternatively, the heterogeneous catalyst is in the form of catalyst particles which are suspended in the reaction mixture and recirculated in the tubular loop reactor (1), wherein the reactor particles comprised in the product outlet stream are separated and recycled to the tubular loop reactor (1).

Accordingly, the tubular loop reactor used in the process of the present invention may comprise a means to separate the etherification catalyst comprised in the product outlet stream and to recycle the separated etherification catalyst to the tubular loop reactor (1). In one alternative, the separated etherification catalyst is recycled by feeding the separated catalyst via recycle stream (5) to the polyol feed inlet (2); see FIGS. 1 and 2. In a different alternative, the separated etherification catalyst is recycled via recycle stream (5) to the tubular loop reactor (1); see FIG. 3. Any conventional method for separating particles from a liquid may be used to separate the heterogeneous catalyst particles from the product stream (4) including, but not limited to, filtering, decanting and centrifugation.

Preferably, static mixers are placed inside the tubular loop reactor. Static mixers ensure maximum mixing between multiple phases of the reaction mixture in the reactor.

Preferably, the tubular loop reactor (1) is a jacketed tubular loop reactor operated under isothermal conditions. A single reactor temperature is desirable since it is believed that a higher than optimum temperature favors the reverse reaction and/or side-reactions.

The process of the present invention is performed in the liquid phase. The process conditions useful in the process of the present invention, also described herein as “polyol etherification conditions”, are well described in the art and can be readily selected by the person skilled in the art; see e.g. U.S. Pat. No. 5,476,971. Accordingly, the reaction temperature may be 20-200° C., preferably is 40-150° C. and more preferably is 60-100° C. The process pressure may be 1-100 barg, preferably is 2-50 barg and more preferably is 3-40 barg.

The polyol comprised in the feed to the process of the present invention is a diol or triol having 2-3 carbon atoms. The term “polyol” as used herein is amply known in the art and describes a hydrocarbon compound comprising more than one hydroxyl group. Preferably, the polyol is selected from the group consisting of glycerol and ethylene glycol. More preferably, the polyol used in the present process is glycerol.

As used herein, the term “alkylation agent” or “alkylating agent” means a hydrocarbon having 2-10 carbon atoms selected from the group consisting of olefins, ketones and alcohols. The hydrocarbon used as alkylation agent may be a straight, branched or cyclic hydrocarbon. Preferably, the alkylation agent is isobutylene.

Most preferably, the polyol is glycerol and the alkylation agent is isobutylene to preferably produce the di-substituted di-ethers 2,3-di-tert-butoxy-1-propanol and 1,3-di-tert-butoxy-l-propanol.

The process described herein includes at least the following embodiments:

### Embodiment 1

A process for the production of polyol alkyl ethers by etherification of a polyol in the presence of an etherification catalyst, wherein the process is performed in a tubular loop reactor (1) comprising a polyol feed inlet (2), at least one alkylation agent feed inlet (3) and a product outlet (4), wherein the polyol is a diol or triol having 2-3 carbon atoms and the alkylation agent is a hydrocarbon having 2-10 carbon atoms selected from the group consisting of olefins, ketones and alcohols.

### Embodiment 2

The process according embodiment 1, wherein the alkylation agent is added to the reaction mixture in the tubular loop reactor at a rate to provide a molar ratio of the hydroxyl groups to alkylation agent in the reaction mixture of 2:1 to 5:1.

### Embodiment 3

The process according embodiment 1 or 2, wherein the reactor comprises multiple alkylation agent feed inlets (3).

### Embodiment 4

The process according to any one of embodiments 1-3, wherein the etherification catalyst is a homogeneous acid catalyst selected from the group consisting of sulfuric acid, sulfonic acid and nitric acid.

### Embodiment 5

The process according to embodiment 4, wherein the homogeneous acid catalyst residue comprised in the product outlet stream is neutralized and the resulting salt is separated from the product outlet stream.

### Embodiment 6

The process according to any one of embodiments 1-3, wherein the etherification catalyst is a porous heterogeneous acid catalyst selected from the group consisting of acidic large-pore zeolite and strong acid ion exchange resin.

### Embodiment 7

The process of embodiment 6, wherein the heterogeneous catalyst is packed in a fixed bed inside the tubular loop reactor (1) or wherein the heterogeneous catalyst is in the form of catalyst particles which are circulated in the tubular loop reactor (1) and wherein the reactor particles comprised in the product outlet stream are separated and recycled to the tubular loop reactor (1).

### Embodiment 8

The process according to any one of embodiments 1-7, wherein static mixers are placed inside the tubular loop reactor.

### Embodiment 9

The process according to any one of embodiments 1-8, wherein the tubular loop reactor (1) is a jacketed tubular loop reactor operated under isothermal conditions.

### Embodiment 10

The process according to any one of embodiments 1-9, wherein the process conditions include a reaction temperature of 20-200° C. and a process pressure of 1-100 barg.

### Embodiment 11

The process according to any one of embodiments 1-10, wherein the polyol in the polyol feed is selected from the group consisting of glycerol and ethylene glycol.

### Embodiment 12

The process according to any one of embodiments 1-11, wherein the alkylation agent is isobutylene.

It is noted that the invention relates to all possible combinations of features described herein, particularly features recited in the claims.

It is further noted that the term ‘comprising’ does not exclude the presence of other elements. However, it is also to be understood that a description on a product comprising certain components also discloses a product consisting of these components. Similarly, it is also to be understood that a description on a process comprising certain steps also discloses a process consisting of these steps.

1. A process for the production of polyol alkyl ethers by etherification of a polyol in the presence of an etherification catalyst, wherein the process is performed in a tubular loop reactor comprising a polyol feed inlet, at least one alkylation agent feed inlet and a product outlet, wherein the polyol is a diol or triol having 2-3 carbon atoms and the alkylation agent is a hydrocarbon having 2-10 carbon atoms selected from olefins, ketones, alcohols or a combination comprising at least one of the foregoing.

2. The process according to claim 1, wherein the alkylation agent is added to the reaction mixture in the tubular loop reactor at a rate to provide a molar ratio of the hydroxyl groups to alkylation agent in the reaction mixture of 2:1 to 5:1.

3. The process according to claim 1, wherein the reactor comprises multiple alkylation agent feed inlets.

4. The process according to claim 1, wherein the etherification catalyst is a homogeneous acid catalyst selected from sulfuric acid, sulfonic acid nitric acid, or a combination comprising at least one of the foregoing.

5. The process according to claim 4, wherein the homogeneous acid catalyst residue comprised in the product outlet stream is neutralized and the resulting salt is separated from the product outlet stream.

6. The process according to claims 1, wherein the etherification catalyst is a porous heterogeneous acid catalyst selected from acidic large-pore zeolite, strong acid ion exchange resin, or a combination comprising at least one of the foregoing.

7. The process of claim 6, wherein the heterogeneous catalyst is packed in a fixed bed inside the tubular reactor or wherein the heterogeneous catalyst is in the form of catalyst particles which are circulated in the tubular loop reactor and wherein the reactor particles comprised in the product outlet stream are separated and recycled to the tubular loop reactor.

8. The process according to claims 1, wherein static mixers are placed inside the tubular loop reactor.

9. The process according to claims 1, wherein the tubular loop reactor is a jacketed tubular loop reactor operated under isothermal conditions.

10. The process according to claims 1, wherein the process conditions include a reaction temperature of 20-200° C. and a process pressure of 1-100 barg.

11. The process according to claim 1, wherein the polyol in the polyol feed is selected from glycerol ethylene glycol, or a combination comprising at least one of the foregoing.

12. The process according to claim 1, wherein the alkylation agent is isobutylene.

13. The process according to claim 1, wherein the process is a one phase or a two phase process.

14. The process according to claim 1, wherein a reactor effluent comprises a single phase.

15. The process according to claim 1, wherein a reactor effluent comprises two phases.

16. The process according to claim 1, further comprising regulating a residence time of materials in the reactor.

17. The process according to claim 1, further comprising regulating molar ratio the hydroxyl groups to alkylation agent in the reactor.
